SEQUENCE LISTING (2 sequences)

SEQ ID NO: 1 - protein, 208 aa, Artificial
  Grafted Light Chain for Fab and Modified Fab
DIQMTQSPSSLSASVGDRVTITCKASQNVGTNVAWYQQKPGKAPKALIYSASFLYSGVPYRFSGSGSGTDFTLTISSLQPEDFATYYCQQYNIYPLTFGQGTKVEIKRTVAAPSVFIFPPSDEQLKSGTASVVCLLNNFYPREAKVQWKVDNALQSGNSGESVTEQDSKDSTYSLSSTLTLSKADYEKHKVYACEVTHQGLSSPVTKS

SEQ ID NO: 2 - protein, 229 aa, Artificial
  Grafted Heavy Chain for Modified Fab
EVQLVESGGGLVQPGGSLRLSCAASGYVFTDYGMNWVRQAPGKGLEWMGWINTYIGEPIYADSVKGRFTFSLDTSKSTAYLQMNSLRAEDTAVYYCARGYRSYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVAHKPSNTKVDKKVEPKSCDKTHTCAA

(19) United States
(12) Patent Application Publication
(10) Pub. No.: US 2004/0091490 A1
(43) Pub. Date: May 13, 2004

(54) Stable pH optimized formulation of a modified antibody

(76) Inventors: Robert Johnson, Skokie, IL (US); Hong Qi, Northbrook, IL (US); Jeffry Borgmeyer, Ballwin, MO (US); Robert Kendall Kessler, Ballwin, MO (US); David Zeng, Wildwood, MO (US)

Correspondence Address: PHARMACIA CORPORATION, GLOBAL PATENT DEPARTMENT, POST OFFICE BOX 1027, ST. LOUIS, MO, 63006, US

(21) Appl. No.: 10/634,581

(22) Filed: Aug. 5, 2003

Related U.S. Application Data
(60) Provisional application No. 60/406,485, filed on Aug. 28, 2002.

(51) Int. Cl. A61K 39/395; C07K 16/46

(52) U.S. Cl. 424/178.1; 530/391.1

(57) ABSTRACT

The present invention is directed to stable formulations of modified antibodies, each comprising an antibody fragment in an aqueous solution covalently attached to at least one nonproteinaceous polymer, such as poly(ethyleneglycol), through a succinimide moiety. CDP870 is an example of one such modified antibody. The formulations of the present invention include a buffer that maintains the solution at a pH of 3.5 to about 6, wherein all components of the modified antibody, including a succinimide ring of the succinimide moiety, are stable.

### CROSS-REFERENCE TO RELATED APPLICATIONS

This application claims the benefit of U.S. Provisional Application Serial No. 60/406,485, filed Aug. 28, 2002.

### FIELD OF THE INVENTION

The present invention relates to stable formulations of modified antibodies, particularly, to modified therapeutic antibody formulations. The invention relates to formulations that stabilize the antibody as well as any and all residues or functional groups covalently attached thereto, including one or more linker moieties. The present invention particularly relates to formulations wherein a succinimide ring structure in a linker of a modified antibody is stabilized.

### BACKGROUND

Antibodies have been identified and developed for use in the diagnosis, prevention, and treatment of many different diseases and disorders. For example, various different types of antibodies with specificity for antigenic determinants of human tumor necrosis factor alpha (TNFα) have been identified, for use in the diagnosis, prevention, and treatment of various diseases associated therewith. Monoclonal antibodies against TNFα have been described in the literature. See, for example, murine monoclonal antibodies disclosed in, Meager et al., Hybridoma 6: 305-311 (1987); Fendly et al., Hybridoma 6: 359-370 (1987); Shimammoto et al., Immunology Letters 17: 311-318 (1988). Complementarity-determining region (“CDR”)-grafted antibodies specific for TNFα are disclosed in Rankin et al., British J. Rheumatology 34: 334-342 (1995). A humanized CDR-grafted modified antibody specific for TNFα, CDP870, is disclosed in international publication number WO 01/94585 A1.

Antibodies to TNFα are particularly useful because TNFα as a pro-inflammatory cytokine that is released by and interacts with cells of the immune system. (WO 01/94585 A1, page 2). TNFα is released by macrophages that have been activated by lipopolysaccarides (LPS) of gram negative bacteria. (Id.) As such, TNFα is believed to be an endogenous mediator involved in the development and pathogenesis of endotoxic shock associated with bacterial sepsis. (Id.) TNFα has also been shown to be up-regulated in a number of human diseases, including chronic diseases such as rheumatoid arthritis, Crohn's disease, ulcerative colitis, and multiple sclerosis. (Id.) For additional references disclosing antibodies to TNFα and their uses, see U.S. Pat. Nos: 6,248,471; 6,528,562; 5,654,407; 6,090,923; and 5,795,697, all of which are incorporated herein by reference. Antibodies to many other antigens implicated in other diseases and disorders are also known.

Antibodies are a type of protein. Like any protein, the biological activity of an antibody, such as its binding affinity, depends upon the conformational integrity of at least a core sequence of amino acids remaining intact while protecting the protein's multiple functional groups from degradation. Chemical and physical instability can each contribute to degradation of an antibody or other protein. Chemical instability can result from deamidation, racemization, hydrolysis, oxidation, beta elimination or disulfide exchange. Physical instability can result from denaturation, aggregation, precipitation, or adsorption, for example. The three most common protein degradation pathways are protein aggregation, deamidation, and oxidation. U.S. Pat. No. 6,267,958, col. 1, lines 29-40, citing Pikal, M. Biopharm. 3(9)26-30 (1990) and Arkawa et al. Pharm. Res. 8(3):285-291 (1991). See also, Stratton, Lewis et al. J. of Pharm. Sci. 90(12):2141-2148 (December 2001).

Modified antibodies include at least one moiety either attached directly to the antibody, or attached indirectly to the antibody through at least one linker. Antibodies are modified for a variety of different reasons, including but not limited to increasing the stability of the antibody, adding a functional group to the antibody to be used to isolate the antibody, or to change the rate at which the antibody is eliminated from a subject after administration thereto. Partial degradation of a linker can result in rendering the moiety ineffective. Complete degradation of a linker results in detachment of a moiety from an antibody.

Therapeutic antibodies enable one to target specific tissues or molecules in a subject, such as cancer cells, specific molecules in defective regulatory pathways, specific molecules that can effect a change in molecules in defective regulatory pathways, and viruses, etc. Some therapeutic antibodies are cleared from a host subject so quickly, after administration thereto, that they do not have enough time to target or effect a change in the targeted tissue or molecules, or in molecules regulated by the targeted molecules. It has been discovered that when antibodies are modified in certain ways, one can significantly increase the residence time of the antibody in a host organism, allowing the antibody significantly more time to reach and bind to its target. One such modification is the covalent attachment of a nonproteinaceous polymer to an antibody, either directly or through a linker. Nonproteinaceous polymers include, but are not limited to, poly(ethyleneglycol), poly(propyleneglycol) (hereinafter, “PEG”), or poly(oxyalkylene) in the manner set forth in U.S. Pat. Nos. 4,640,835; 4,496,689; 4,301,144; 4,670,417; 4,791,192; all of which are incorporated by reference herein. For example, it has been discovered that site-specific attachment of at least one PEG residue, to a Fab′ hinge region of an antibody fragment increases the in vivo half-life of an antibody fragment without loss of antigen-binding affinity, compared to unmodified antibody fragments and to antibody fragments modified by random attachment of PEG residues thereto. Chapman, Andrew P. et al., Nature Biotechnology 17:780-783 (August 1999).

Various linkers have been used to connect one or more molecules of PEG or other nonproteinaceous polymers to antibodies. For example, maleimide groups have been used to link PEG to antibodies or to antibody fragments. When the maleimide is not a leaving group, it generally becomes at least part of a linker between the PEG residue(s) and the antibody or antibody fragment. In such cases, the maleimide group is converted into a succinimide moiety. The vendor of substrates for producing PEGylated antibodies with succinimide based linkers, Shearwater Corporation, states that mPEG-MAL, a brand name for a reactive substrate consisting of maleimide covalently linked to a PEG residue, and mPEG2-MAL, a brand name for a reactive substrate comprising maleimide covalently linked to two PEG residues through a lysine residue, claims the two substrates readily couple to thiol groups of proteins (or antibodies), producing bioactive PEG-protein conjugates. (Shearwater Corp. Catalog 2001: Polyethylene Glycol and Derivatives for Biomedical Applications, p. 8, col. 2). For an illustration of the use of PEG-maleimide and branched PEG₂-maleimide to attach PEG residues to an antibody fragment through a succinimide moiety, either randomly or in a site-specific manner, see Chapman, supra. For additional methods for attachment of poly(ethyleneglycol) polymer(s) to antibody fragments through a succinimide moiety, see U.S. Pat. Nos.: 6,113,906; 5,919,455; 5,643,575; and 5,932,462; and in EP 788 515, all of which are incorporated by reference herein.

CDP870, described in WO 01/94585 A1 (filed by Celltech R & D Ltd.), is an example of a modified therapeutic antibody, with two PEG residues covalently attached to the antibody through a linker comprising a succinimide moiety in the form of a succinimide ring, and a lysine residue. Specifically, CDP870 comprises an antibody fragment with a light chain and a heavy chain derived from a mouse monoclonal antibody having specificity for human TNFα, covalently linked to a succinimide moiety that is in turn covalently linked to a lysine residue covalently linked to two methoxypropyl(ethyleneglycol) polymers (of approximately 20,000 Da each). (Id., page 9, line to page 10, line 2 and FIG. 13). The antibody sequences of CDP870 are vulnerable to degradation, as are any amino acid sequences.

Succinimide ring structures, such as are present in many of the modified antibodies described above, including CDP870, have been found to open and, at times, degrade, in the presence of a solution at high pH. Ishi, Yoshiharu, et al., Biophys. J. 50:75-80 (July 1986).

U.S. Pat. No. 6,171,586 B1 by Zanthe Lam et al. discloses an aqueous antibody formulation comprising an antibody in a pH-buffered solution having “a pH in the range from about 4.5 to about 6.0, preferably from about 4.8 to about 5.5.” (Column 22, lines 18-21) The concentration of buffer in the formulation depends upon the buffer and the desired isotonicity. (Id., col. 22, lines 25 to 28) However, the formulation also includes a polyol, which may act as a stabilizer and also acts as a tonicifier. (Id., col. 22, lines 31-32). A surfactant is also included in the formulation. (Id., col . 22, line 49). The patent also discloses that the formulation preferably “does not contain a tonicifying amount of a salt such as sodium chloride, as this may cause the antibody to precipitate and/or may result in oxidation at low pH.” (Id., col. 22, lines 32 to 36; see, also, language of claim 1). The '586 patent discloses that many different types of antibodies can be stabilized in the formulation described immediately above.

None of the antibodies disclosed in the '586 patent are described as being suitable for stabilization in the solution are modified with nonproteinaceous polymers, much less with such polymers linked to an antibody through a linker containing a succinimide ring. The '586 patent also indicates that no antibody formulation buffered at a low pH would be stable if it contained a tonicifying amount of a salt, such as sodium chloride (see above).

It is an object of the present invention to provide a means for stabilizing the succinimide ring structure of modified antibody formulations comprising a succinimide moiety. It is a further object of the present invention to provide stable formulations of such modified antibodies, including, CDP870.

### BRIEF SUMMARY OF THE INVENTION

The present invention relates to an aqueous antibody formulation comprising (a) a modified antibody comprising an antibody fragment and at least one nonproteinaceous polymer covalently attached to the antibody fragment through a linker comprising a succinimide moiety; and (b) a buffer at pH of about 3.5 to about 6.0. In a particularly preferred embodiment, the modified antibody is CDP870.

The present invention also relates to a method of treatment or prophylaxis of an inflammatory disease steps comprising: (a) providing the formulation of the present invention, described above, wherein the modified antibody has an affinity for an inflammatory disease antigen; and (b) delivering a pharmaceutically effective dose of the formulation to a subject in order to treat or prevent an inflammatory disease.

The formulation and method of the present invention provide a useful means for the treatment or prevention of diseases associated with TNFα, including but not limited to inflammatory diseases, such as endotoxic shock associated with bacterial sepsis or chronic diseases such as rheumatoid arthritis, Crohn's disease, ulcerative colitis, and multiple sclerosis. Additional diseases or conditions associated with TNFα that could be treated with the formulation of the present invention include: primary biliary cirrhosis; Myelodysplastic syndrome; chronic variable immunodefficientcy; treatment refractory sarcoidosis; diffuse lung disease, such as pulmonary fibrosis that is idiopathic or secondary to RA, or acute interstitial pneumonitis; vasculitis, such as Wegeners vasculitis, polyarteritis nodosa, temporal arteritis, IgA nephropathy (Henoch-Schonlein Purpura); crescentic nephritis-juvenile treatment resistant uveitis; adult treatment resistant uveitis; primary sclerosing cholangitis, alcohol induced hepatitis, ulcerative colitis, inflammatory skin diseases, such as bullous pemhigoid, and pemphigus vulgaris; and polyositis (dermatomyositis). Other advantages of the present invention will become apparent upon reviewing the remainder of the present specification, below.

### BRIEF DESCRIPTION OF THE DRAWING(S)

FIG. 1 is a plot of results of measurements of % acidic species in a formulation of 170 mg/ml CDP870, 125 mM NaCl, 10 mM Sodium Acetate at various pH's stored at 25° C. in vials, over a period of twelve (12) weeks.

FIG. 2 is a plot of results of measurements of % dePEGylation in the same formulation described for FIG. 1, above, after storage at 40° C. in vials, over a period of twelve (12) weeks.

### DETAILED DESCRIPTION OF THE INVENTION

The term “antibody,” as used herein, covers monoclonal antibodies (including full length monoclonal antibodies), polyclonal antibodies, multispecific antibodies (e.g., bispecific antibodies), antibody fragments, and modified antibodies, so long as they exhibit the desired biological activity.

“Antibody fragments” comprise a portion of a full length antibody, generally the antigen binding or variable region thereof. Examples of antibody fragments include Fab, Fab′, F(ab′)₂, and Fv fragments, diabodies, linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) and Marks et al., J. Mol. Biol. 222:581-597 (1991), for example.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad Sci. USA 81:6851-6855 (1984)).

The term “hypervariable region,” as used herein, refers to the amino acid residues of an antibody which are responsible for antigen-binding. The hypervariable region comprises amino acid residues from a “complementary determining region” or “CDR” (i.e. residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e. residues 26-32 (L1), 50-52 (L2) and 91-96 (L3) in the light chain variable domain and 26-32 (H1), 53-55 (H2) and 96-101 (H3) in the heavy chain variable domain; Chothia Lesk J. Mol. Biol. 196:901-917 (1987)).

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. For further details, see Jones et al., Nature 321:522-525 (1986); Riechmann et al, Nature 332:323-329 (1988); and Presta, Curr. Op. Struct. Biol. 2:593-596 (1992).

The term “modified antibody” as used herein refers to an antibody covalently attached to at least one modifying group. The modifying group can be any one of a number of different functional or inert groups, including, but not limited to halogens, thiols, other non-proteinaceous residues, or one or more amino acid residues in combination with at least one non-proteinaceous residues. The modifying group can be covalently attached directly to the antibody or, when the antibody is an antibody fragment, to the antibody fragment, or be indirectly attached thereto through a linker.

The formulations of the present invention comprise a modified antibody, comprising an antibody fragment covalently attached to at least one type of non-proteinaceous residue, a nonproteinaceous polymer. The antibody fragment is preferably a therapeutic antibody fragment, although, it can be any type of antibody fragment. The antibody fragment preferably comprises a heavy chain and a light chain, such as the antibody fragment component of CDP870, a therapeutic antibody fragment containing at least one CDR having specificity for human TNFa. The light chain of CDP870 comprises an amino acid sequence identified by SEQ ID NO:1, while the heavy chain comprises an amino acid sequence identified by SEQ ID NO:2. These two sequences correspond to SEQ ID NO: 113 and SEQ ID NO: 115 of WO 01/94585, respectively. The modified antibody is preferably CDP870.

At least one nonproteinaceous polymer covalently attached to the modified antibody is preferably polyethylene glycol, polypropylene glycol, polyoxyalkylene, or a derivative thereof. The nonproteinaceous polymer is more preferably poly(ethyleneglycol) or a derivative thereof, such as a methoxypoly(ethyleneglycol) polymer. At least one nonproteinaceous polymer is preferably one which increases the residence time of the modified antibody in a host subject, after administration thereto, compared to the residence time of an antibody without any nonproteinaceous polymer component. Each nonproteinaceous polymer is preferably 5,000 to about 50,000 Daltons (Dal), more preferably about 10,000 to about 40,000 Dal, even more preferably about 15,000 to about 30,000 Dal, most preferably about 20,000 Dal. in length.

In the modified antibody formulations of the present invention, the at least one nonproteinaceous polymer is covalently attached to the antibody fragment through a linker comprising a succinimide moiety. CDP870 is one such modified antibody. The structure of CDP870 is illustrated in formula I, below:

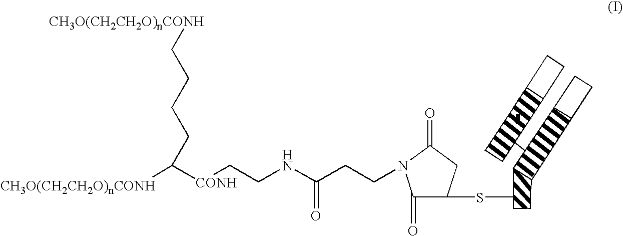

The C-terminal end of the heavy chain of the Fab fragment, shown on the right side of formula I, above, includes a modified hinge region that is covalently linked to a succinimide moiety through a cysteine residue of the antibody fragment. Two methoxypolytethyleneglycol) polymers, each having a molecular weight of approximately 20,000 Da, are covalently attached to a lysine residue that is linked to the antibody fragment by covalent attachment to the succinimide moiety.

The formulation of the present invention further comprises a buffer that maintains the pH of the formulation at a pH at which the modified antibody is stable. More particularly, the formulation is preferably at a pH at which no more than a minimal percent of the succinimide rings of the linkers of all the modified antibody molecules in the formulation are hydrolyzed over time. Preferably, less than about 15%, more preferably less than about 10%, even more preferably less than about 5% of the succinimide rings are hydrolyzed after at least one (1) year of storage at room temperature. The pH of the formulation is preferably at about pH 3.5 to about 6, more preferably at about pH 4 to about 5.5, more preferably at about pH 4.5 to about pH 5, even more preferably about pH 4.8.

In a preferred embodiment of the formulation of the present invention, the buffer is preferably a buffer that can maintain the pH of the solution at the desired pH range over a long period of time, preferably for at least one year, even more preferably for at least two years. Suitable buffers for use in the formulation of the invention include, but are not limited to, acetate, histidine, citrate, lactate, or succinate buffers.

The formulation of the present invention is also preferably stable, as assayed using a standard aggregation assay. Specifically, less than about 5%, preferably less than about 3%, even more preferably less than about 1% of the modified antibody is preferably present in aggregated form after one (1) year of storage of the formulation at 25° C.

In another preferred embodiment of the formulation of the present invention, the modified antibody is present at a concentration that is suitable for delivery of a therapeutically or prophylactically effective dose in a volume suitable for injection by parenteral means. The term “suitable for injection”, as used herein, refers to a solution that is not so viscous that it cannot be injected into a subject, using any means, and not so dilute that delivery of too much volume would be required to make practical delivery of the formulation by such means. The modified antibody concentration in the formulation is preferably about 50 mg/ml to about 300 mg/ml, more preferably about 100 mg/ml to about 250 mg/ml, even more preferably about 150 mg/ml to about 220 mg/ml. The formulation of the present invention is preferably suitable for parenteral injection.

In another embodiment, the formulation of the present invention is isotonic. When a non-isotonic solution is administered to a subject and comes into contact with living cells, the cells will tend to swell or shrink, due to osmotic pressure, and sometimes even burst. In contrast, cells neither swell nor shrink in the presence of an isotonic solution. Isotonicity can be achieved by using an isotonic amount of at least one tonicifier. Suitable tonicifiers for use in the formulation of the present invention include salts, such as sodium chloride, and polyols, nonreducing sugars, such as sucrose or trehalose. In one embodiment, the formulation of the present invention contains a tonicifying concentration of sodium chloride, or of sodium chloride and sodium chloride equivalents. When the formulation is designed to be administered to a human subject, the tonicifying amount is preferably about 135 mM to about 175 mM sodium chloride or sodium chloride and sodium chloride equivalents.

In another embodiment, the formulation of the present invention is isotonic and contains a polyol and less than a tonicifying amount of sodium chloride, as described in U.S. Pat. No. 6,171,586 B1, col. 22, lines 31-59, incorporated herein by reference.

In yet another embodiment, the modified antibody further comprises a surfactant, preferably a nonionic surfactant. Nonionic surfactants suitable for use in the formulations of the present invention include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.). The surfactant is more preferably polysorbates 20 or 80, or a mixture of polysorbates 20 and 80.

In another embodiment, the formulation of the present invention further comprises a preservative. Suitable preservatives for inclusion in the formulation of the present invention include, but are not limited to, mercury-containing substances such as phenylmercuric salts (e.g., phenylmercuric acetate, borate and nitrate) and thimerosal; stabilized chlorine dioxide; quaternary ammonium compounds such as benzalkonium chloride, cetyltrimethylammonium bromide and cetylpyridinium chloride; imidazolidinyl urea; parabens such as methylparaben, ethylparaben, propylparaben and butylparaben, and salts thereof; phenoxyethanol; chlorophenoxyethanol; phenoxypropanol; chlorobutanol; chlorocresol; phenylethyl alcohol; disodium EDTA; and ascorbic acid and salts thereof.

The present invention is also directed to a method of treatment or prophylaxis of a disease, comprising providing the modified antibody formulation of the invention, described herein above, and delivering a pharmaceutically effective dose of the formulation to a subject to treat or prevent a disease associated with a disease antigen. The disease antigen is preferably TNFα. The disease is preferably one associated with TNFα, such as primary biliary cirrhosis; Myelodysplastic syndrome; chronic variable immunodefficientcy; treatment refractory sarcoidosis; diffuse lung disease, such as pulmonary fibrosis that is idiopathic or secondary to RA, or acute interstitial pneumonitis; vasculitis, such as Wegeners vasculitis, polyarteritis nodosa, temporal arteritis, IgA nephropathy (Henoch-Schonlein Purpura); crescentic nephritis juvenile treatment resistant uveitis; adult treatment resistant uveitis; primary sclerosing cholangitis, alcohol induced hepatitis, ulcerative colitis, inflammatory skin diseases, such as bullous pemhigoid, and pemphigus vulgaris; polyositis (dermatomyositis); or an inflammatory disease, such as endotoxic shock associated with bacterial sepsis or a chronic disease such as rheumatoid arthritis, Crohn's disease, ulcerative colitis, and multiple sclerosis. The disease treated according to the present method is, even more preferably, rheumatoid arthritis. The subject is preferably a mammal, more preferably a human being. When the disease antigen is TNFα and the subject is a human being, the modified antibody formulation is preferably a CDP870 formulation.

The present invention is further illustrated by the following examples. These examples are intended to be illustrative of the invention and should not be used to limit or restrict its scope.

### EXAMPLES

### Example 1-Production of Formulations of CDP870

Formulations of CDP870 for use in the experiments described in Example 2, below, were produced from solutions of CDP870 obtained from CellTech R&D Ltd (Berkshire, Great Brittain). The starting formulation composition was 200 mg/ml CDP870 in 125 mM NaCl, and 50 mM sodium acetate (pH 5.5). Each such solution was treated by either dialysis combined with a concentrating spin filtration procedure, or by diafiltration in combination with ultrafiltration. The first step of each combined procedure was included in order to enable one to change the buffer and other solution conditions. The second step of each procedure was included in order to allow one to produce a more concentrated formulation of CDP870, one that could be more pharmaceutically effective when administered to a human being by parenteral injection.

### Example 2-Stability of CDP870 Formulations at Different pH's

The stability of four formulations of CDP870, varying in pH from 4.15 to 5.5 was tested, as follows. Each formulation of CDP870 had a concentration of 160 mg/ml of CDP870, 10 mM Sodium Acetate, and 0.125 M Sodium Cloride, with a pH of: (1) 4.15, (2) 4.5, (3) 5.0, and (3) 5.5, respectively. Aliquots of 0.2 ml were placed in 2 ml. Schott glass vials, stoppered with 13 mm Diakyo Flurotec serum stoppers, and stored at 5° C., 25° C., or 40° C. The contents of each vial were then withdrawn for stability testing, at time points set forth in Table 1, below. Additional vials of the same four formulations were stored at the same three temperatures, to be tested after the first 12 weeks, at six month intervals beginning six months after time zero, and extending to three years after time zero (0).

In addition to the vials described above, two “contingency” vials of each formulation were placed at 5° C. The contents of one vial were used to determine the concentration of CDP870 in the formulations tested, by UV analysis. The other vial was diluted, aliquoted, and frozen for use as controls.

Each sample was tested for stability by examining appearance, by conducting size exclusion (“SEC”) analysis using HPLC, by cation exchange HPLC (CEX), by isoelectric focusing (“IEF”), by peptide mapping (at 0, 3 months, and 24 months), and by testing the pH of the formulation (at 0, 8 weeks, and 12 weeks). The results of the SEC, IEF, and CEX assays are summarized in Table 1, below:

TABLE 1

| | | | | | | CEX |
|---|---|---|---|---|---|---|
| | | | | | | Total % |
| Formu- | | | SEC | SEC | IEF | Acidic |
| lation | | | Area % | Area % | % | Species |
| # | Temper- | Time | Aggre- | Fab/ | Acidic | Two |
| (pH) | ature | (week) | gate | DiFab | Species | Peaks |
| # 1 | 5° C. | 0 | 1.2 | 0.2 | 11 | 18 |
| (pH 4.15) | | 4 | 1.3 | 0.2 | 13 | — |
| | | 12 | 1.5 | 0.3 | 10 | 21 |
| | 25° C. | 1 | 1.4 | 0.4 | 13 | — |
| | | 2 | 1.3 | 0.5 | 15 | 20 |
| | | 3 | 1.3 | 0.9 | 14 | — |
| | | 4 | 1.4 | 0.9 | 15 | — |
| | | 8 | 1.6 | 1.5 | 14 | — |
| | | 12 | 1.7 | 2.4 | 17 | 25 |
| | 40° C. | 1 | 1.9 | 1.6 | 17 | — |
| | | 2 | 2.4 | 3.2 | 18 | 25 |
| | | 3 | 2.8 | 5.0 | 19 | — |
| | | 4 | 2.8 | 6.0 | 20 | — |
| | | 8 | 3.6 | 9.2 | 26 | — |
| | | 12 | 4.6 | 14.3 | 27 | 37 |
| #2 | 5° C. | 0 | 2.2 | 0.2 | 14 | 20 |
| (pH 4.5) | | 4 | 2.3 | 0.2 | 13 | — |
| | | 12 | 2.8 | 0.3 | 12 | 22 |
| | 25° C. | 1 | 2.3 | 0.3 | 15 | — |
| | | 2 | 2.1 | 0.5 | 15 | 24 |
| | | 3 | 2.0 | 0.6 | 17 | — |
| | | 4 | 2.1 | 0.7 | 17 | — |
| | | 8 | 2.7 | 1.1 | 20 | — |
| | | 12 | 2.8 | 1.6 | 19 | 27 |
| | 40° C. | 1 | 2.4 | 1.1 | 14 | — |
| | | 2 | 2.5 | 2.2 | 20 | 26 |
| | | 3 | 2.6 | 3.2 | 22 | — |
| | | 4 | 2.6 | 4.1 | 23 | — |
| | | 8 | 3.7 | 6.4 | 27 | — |
| | | 12 | 4.1 | 9.8 | 37 | 38 |
| #3 | 5° C. | 0 | 3.3 | 0.3 | 13 | 18 |
| (pH 5.0) | | 4 | 3.2 | 0.3 | 15 | — |
| | | 12 | 3.6 | 0.3 | 14 | 23 |
| | 25° C. | 1 | 3.2 | 0.3 | 16 | — |
| | | 2 | 3.2 | 0.5 | 16 | 24 |
| | | 3 | 3.1 | 0.6 | 17 | — |
| | | 4 | 3.0 | 0.7 | 19 | — |
| | | 8 | 3.3 | 1.0 | 21 | — |
| | | 12 | 3.4 | 1.4 | 23 | 31 |
| | 40° C. | 1 | 3.3 | 1.1 | 22 | — |
| | | 2 | 3.2 | 1.8 | 24 | 31 |
| | | 3 | 3.1 | 2.6 | 24 | — |
| | | 4 | 3.2 | 3.2 | 28 | — |
| | | 8 | 3.9 | 4.9 | 35 | — |
| | | 12 | 4.6 | 8.2 | 40 | 40 |
| #4 | 5° C. | 0 | 3.9 | 0.3 | 15 | 19 |
| (pH 5.5) | | 4 | 3.8 | 0.3 | 17 | — |
| | | 12 | 4.2 | 0.3 | 22 | 25 |
| | 25° C. | 1 | 3.8 | 0.5 | 20 | — |
| | | 2 | 3.8 | 0.6 | 21 | 28 |
| | | 3 | 3.5 | 0.7 | 23 | — |
| | | 4 | 3.5 | 0.8 | 27 | — |
| | | 8 | 3.9 | 1.2 | 31 | — |
| | | 12 | 3.8 | 1.6 | 41 | 43 |
| | 40° C. | 1 | 3.7 | 1.2 | 29 | — |
| | | 2 | 3.7 | 2.1 | 38 | 41 |
| | | 3 | 3.7 | 2.9 | 44 | — |
| | | 4 | 3.8 | 3.5 | 48 | — |
| | | 8 | 4.6 | 5.5 | 59 | — |
| | | 12 | 5.2 | 8.6 | 69 | 57 |

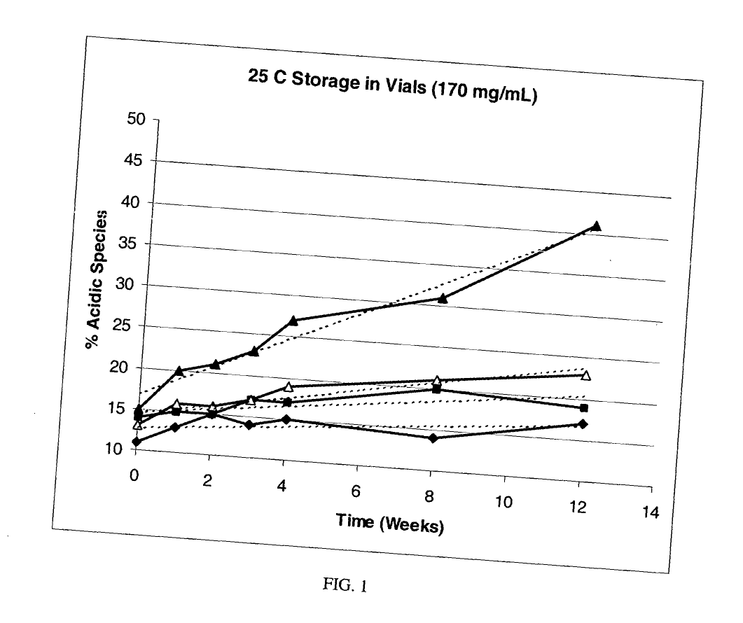

The IEF assay was included in order to detect one acidic species in a formulation over time, one way of detecting the break-down of the succinimide ring of a modified antibody, such as CDP870. IEF assay results from the samples of each formula stored at 25° C., shown in Table 1, above, are illustrated in FIG. 1. In FIG. 1, the % Acidic Species measured by IEF are plotted against time, in weeks, for the first 12 weeks of the study. The following symbols were used to indicate data plotted from each pH formulation: solid diamond (♦) symbols for the pH 4.1 formulation data points, solid square (▪) symbols for the pH 4.5 formulation data points, hollow triangle (Δ) symbols for. the pH 5.0 formulation data points, and solid triangle (▴) for pH 5.5 data points. Data points for each formulation were connected by solid lines, while a dashed line was used to show the trend line of the data for each formulation.

FIG. 1 clearly shows that the % of acidic species in each formulation tested increased over time, as the pH of the formulation increased, although, the increase in % acidic species was most pronounced in the formulation with a pH of 5.5. However, the % acidic species in the pH 5.5 formulation was considerably lower than that found at higher pH's. The pH 4.1, 4.5, and 5.0 formulations were slightly different from one another, with the pH 4.1 formulation found to produce the lowest % acidic species of all the formulations at each time point. However, all three of the lowest pH formulations tested produced such a low percent of acidic species in the time period studied.

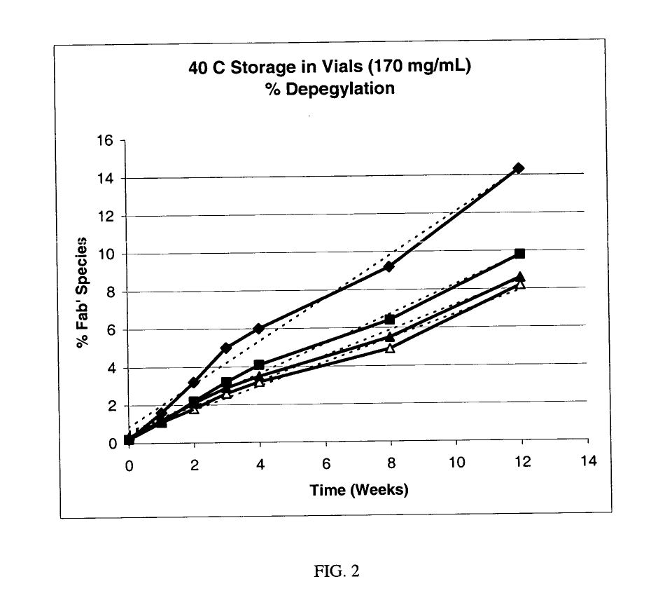

The SEC analysis results were used to determine the % Fab′ species, and to thereby quantify the degree of dePEGylation of samples at each temperature. The results for the formulations at each of the four pH's stored at 40° C. are illustrated in FIG. 2, a plot of % Fab′ species over time during the first 12 weeks of the study. The same symbols were used to indicate data corresponding to each of the same formulations illustrated in FIG. 1, discussed immediately above. Trend lines were, similarly shown, using dashed lines for each data series.

This last study showed that the % Fab′ species increased over time in each vial tested, and there appeared to be an inverse relationship between pH and the % Fab′ present at each time point after the first week, with the difference becoming greater over time. However, there was little difference in the % Fab′ species present at any given time point for samples of the three formulations with the highest pH tested, i.e. those with a pH of 5.0, 5.5, and 4.5. The formulation with a pH of 4.1 showed a significantly greater increase in % Fab′ species over time, indicating the greatest amount of dePEGylation of all the samples tested, at each time point. with each time point tested with the greatest rate of increase appearing in the formulation with a pH of 4.1.

The results of the peptide mapping assay of the four formulations described above are summarized in Table 2, below. The results are all expressed in terms of % Oxidation (M3) at 12 weeks. Note that % Oxidation is a measure of the amount of methionine, one of the amino acid residues of the antibody fragment of CDP870, that is oxidized to the sulfoxide form.:

TABLE 2

| Temperature | pH 4.1 | pH 4.5 | pH 5.0 | pH 5.5 |
|---|---|---|---|---|
| 40° C. | 13.6 | 105 | 66 | 4.2 |
| 25° C. | 6.7 | 36 | 29 | 2.7 |
| 5° C. | 1.6 | 17 | 22 | 2.2 |

The results above show that a pH 5.5 formulation of CDP870 does not undergo a significant amount of dePEGylation or oxidation, even after 12 weeks at room temperature. The pH 5.5 formulation does appear to produce some acidic species over time compared to lower pH formulations. However, lower pH solutions tend to be more poorly tolerated upon injection into mammalian subjects compared to solutions having a pH closer to a neutral pH.

In view of the above, it was decided to use CDP870 formulations with a pH of about 5.5. After further testing, a preferred formulation of 200 mg/ml CDP870 in 50 mM Sodium Acetate (pH 5.5) and 125 mM Sodium Chloride as identified.

We claim:

1. A stable modified antibody formulation comprising:
a modified antibody in an aqueous solution, the modified antibody comprising an antibody fragment and at least one nonproteinaceous polymer covalently attached to the antibody fragment through a linker comprising a succinimide moiety; and
a buffer that maintains the aqueous solution pH at about 3.5 to 6.

2. The formulation of claim 1, wherein the modified antibody has a specificity for human TNFα.

3. The formulation of claim 1, The method of claim 1, wherein the at least one nonproteinaceous polymer is at least one poly(ethyleneglycol) polymer.

4. The formulation of claim 3, wherein the at least one poly(ethyleneglycol) polymer is at least two methoxypoly(ethyleneglycol) polymers.

5. The formulation of claim 4, wherein the at least two methoxypoly(ethyleneglycol) polymers are covalently attached to a lysine residue linked to the succinimide moiety.

6. The formulation of claim 5, wherein the modified antibody is CDP870.

7. The formulation of claim 1, wherein the modified antibody is present at a concentration of about 50 mg/ml to about 300 mg/ml.

8. The formulation of claim 1, wherein less than about 5% of a succinimide ring of the succinimide moiety is present in hydrolyzed form.

9. The formulation of claim 1, wherein less than about 5% of the modified antibody is present in an aggregated form.

10. The formulation of claim 1, wherein the buffer is an acetate buffer.

11. The formulation of claim 1, wherein the formulation is isotonic.

12. The formulation of claim 10, wherein the formulation further comprises a tonicifying amount of sodium chloride.

13. A stable CDP870 formulation, comprising CDP870 in an aqueous solution and a buffer that maintains the pH of the solution from about 3.5 to about 6.0.

14. The formulation of claim 13, wherein the formulation is isotonic.

15. The formulation of claim 14, wherein the formulation further comprises a tonicifying amount of a salt.

16. The formulation of claim 15, wherein the salt is sodium chloride.

17. The formulation of claim 13, wherein the modified antibody is present at a concentration of about 50 mg/ml to about 300 mg/ml.

18. A method of treatment or prophylaxis of a disease, comprising steps of:
a. providing a formulation comprising a modified antibody in an aqueous solution and a buffer that maintains the formulation pH at about 3.5 to about 6.0, the modified antibody comprising an antibody fragment and at least one nonproteinaceous polymer covalently attached to the antibody fragment through a succinimide moiety, wherein the antibody has an affinity for an inflammatory disease antigen; and
b. delivering a pharmaceutically effective dose of the formulation to a subject to treat or prevent a disease associated with a disease antigen.

19. The method of claim 18, wherein the disease antigen is TNFα.

20. The method of claim 19, wherein the disease is rheumatoid arthritis.

21. The method of claim 18, wherein the buffer is acetate buffer.

22. The method of claim 18, wherein the at least one nonproteinaceous polymer is at least one poly(ethyleneglycol) residue.

23. The method of claim 22, wherein the at least one poly(ethyleneglycol) residue is at least one methoxypoly(ethyleneglycol) residue.

24. The method of claim 23, wherein the modified antibody is CDP870.

25. The method of claim 18, wherein the modified antibody is present in the formulation at a concentration of about 50 mg/ml to about 300 mg/ml.

26. The method of claim 18, wherein the dose of the formulation is delivered to the subject parenterally.

27. The method of claim 18, wherein the subject is a mammal.

28. The method of claim 27, wherein the subject is a human being.
